(19) United States
(12) Patent Application Publication
(10) Pub. No.: US 2025/0113960 A1
(43) Pub. Date: Apr. 10, 2025

(54) Steam Port Device

(71) Applicant: David McKee, Doraville, GA (US)

(72) Inventor: David McKee, Doraville, GA (US)

(21) Appl. No.: 18/520,683

(22) Filed: Nov. 28, 2023

Related U.S. Application Data
(60) Provisional application No. 63/587,754, filed on Oct. 4, 2023.

(51) Int. Cl. A47L 11/34; A47L 11/40; A61L 2/07; A61L 2/26; B60S 3/00

(52) U.S. Cl. CPC A47L 11/34; A47L 11/4002; A47L 11/4072; A47L 11/4086; A61L 2/07; A61L 2/26; B60S 3/008; A61L 2202/11; A61L 2202/16; A61L 2202/17

(57) ABSTRACT

The present invention relates to a steam port device that allows the user to properly clean and disinfect a vehicle. The device is comprised of a body, at least one motor fixed inside of the body, and at least one steam wand. The user can select the different steaming options to disinfecting a vehicle. Further, the body is comprised of a digital touch screen and payment panel to allow the user easy accessibility when operating the device. In addition, the steam wand is comprised of a trigger and a hose which allows the user to clean and disinfect the interior of a vehicle with improved accessibility so that the user does not need to strain themselves during the cleaning process.

### CROSS-REFERENCE TO RELATED APPLICATION

The present application claims priority to, and the benefit of, U.S. Provisional Application No. 63/587,754, which was filed on Oct. 4, 2023, and is incorporated herein by reference in its entirety.

### FIELD OF THE INVENTION

The present invention relates generally to the field of steaming devices. More specifically, the present invention relates to a steam port device that is comprised of a body, at least one motor fixed inside of the body, and at least one steam wand. The device allows a user to clean and disinfect the interior of a vehicle with improved accessibility so that the user does not need to strain themselves during the cleaning process. Accordingly, the present disclosure makes specific reference thereto. Nonetheless, it is to be appreciated that aspects of the present invention are also equally applicable to other like applications, devices, and methods of manufacture.

### BACKGROUND

Many vehicles are used day to day for work commutes, leisure trips, and running errands. Over time, the interior of the vehicle can become dirty from mud, dirt, food, liquids, or other debris. This can cause discomfort for the driver while using the vehicle. The buildup of said debris can also cause bacteria or other harmful pathogens to exist inside the vehicle. The bacteria can cause sickness if not taken care of properly. In addition, a messy vehicle interior is unsightly and a vehicle owner may find the messy vehicle interior embarrassing. The user may also be incapable of bending over to hard-to-reach places within the vehicle while cleaning, thereby causing strain to their back and other muscles in an attempt to do so.

When cleaning vehicles, individuals normally require multiple tools such as a shop vacuum, wet wipes, and cleaning solutions to effectively clean the entire interior of a vehicle. Cleaning the interior of the vehicle can be incredibly difficult, frustrating, and time consuming. Individuals may also lack the means to clean their vehicle via steam, ultimately leaving behind germs and bacteria that can be spread between passengers during a trip. Even if an individual does have access to a steam tool, most individuals do not know the proper duration of a steam cycle to ensure that all harmful airborne pathogens are properly taken care of.

Therefore, there exists a long-felt need in the art for an improved steam port device. There also exists a long-felt need in the art for a steam port device that provides users with a commercial steam cleaning system capable of being used to clean and disinfect the interior of a vehicle. In addition, there exists a long-felt need in the art for a steam port device that allows users to use a steam wand to dispense the steam into the vehicle for disinfecting and cleaning the vehicle interior. Further, there exists a long-felt need in the art for a steam port device that has multiple attachments in order to properly reach certain areas of the vehicle as well as properly disinfect different interior vehicle surfaces.

The subject matter disclosed and claimed herein, in one embodiment thereof, comprises a steam port device. The device is comprised of a body, at least one motor fixed inside of the body, and at least one steam wand. In the preferred embodiment, the body is comprised of at least one digital touch screen, at least one locking mechanism, at least one button, at least one payment panel, at least one water inlet, and at least one power cable. Further, the steam wand is comprised of at least one hose, at least one trigger, at least one wand grip, at least one hose connector, and at least one wand storage which allows the user to clean and disinfect the interior of a vehicle with improved accessibility so that the user does not need to strain themselves during the cleaning process.

In this manner, the steam port device of the present invention accomplishes all of the foregoing objectives and provides an improved means to disinfect and clean a vehicle. Further, the steam port device has an easy-to-read digital touch screen as well as multiple payment methods to allow the user to accessibly use the device. In addition, the steam port device has a steam wand with a wand grip and trigger which allows the user to clean and disinfect their car with little risk of injury.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a steam port device that provides users with a commercial steam cleaning system capable of being used to clean and disinfect the interior of a vehicle. The system includes a steam wand and hose for improved accessibility such that a user does not need to strain themselves during the cleaning process. The system is comprised of a device primarily comprised of a body, at least one motor fixed inside of the body, and at least one steam wand. In the preferred embodiment, the body is comprised of at least one digital touch screen, at least one locking mechanism, at least one button, at least one payment panel, at least one water inlet, and at least one power cable. In a differing embodiment, the body may be comprised of a plurality of wheels. In this manner, the device may be rolled around by the user and made easily transportable.

The digital touch screen may be any touch screen known in the art that allows the user to access the different steaming options. In this manner, the user can easily see and select the different steaming options available to them when deciding which option is best for disinfecting their vehicle. The locking mechanism may be any locking mechanism known in the art that allows the body panel to be locked. In this manner, the locking mechanism can keep thieves from being able to steal money from the device. The button is fixed to the front of the body and allows the user to start the device.

The water inlet may be any inlet known in the art that can properly connect a water supply to the device. In this manner, the user can connect a water supply to the device to provide the device with water to produce the steam used for disinfecting and cleaning the vehicle. The power cable may be any power cable known in the art that can properly supply the correct amount of energy needed for the device to maintain proper function.

The payment panel is comprised of at least one credit card reader, at least one bill receiver, and at least one coin slot. The credit card reader can be any credit card reader known in the art that allows the user to make a payment. The bill receiver and the coin slot are fixed to the front of the payment panel. In this manner, the user has the option of three separate forms of payment when using the device.

The motor may be any motor known in the art that can generate enough heat to produce steam from the water supply. The steam wand is comprised of at least one hose, at least one trigger, at least one wand grip, at least one hose connector, and at least one wand storage. The hose carries the steam to the steam wand. In the preferred embodiment, the hose may be a rubber material that is preferably heat-resistant to protect against the high temperatures of the steam or any hose of the like. The hose attaches to the body of the device through a hose connector. The hose connector secures the hose to the body of the device and may be any connector known in the art that is heat resistant.

Accordingly, the steam port device of the present invention is particularly advantageous as it provides users with a commercial steam cleaning system capable of being used to clean and disinfect the interior of a vehicle that includes a steam wand and hose for improved accessibility so that the user does not need to strain themselves during the cleaning process. In this manner, the steam port device overcomes the limitations of existing steam cleaners known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of a steam port device of the present invention in accordance with the disclosed architecture; and

FIG. 2 illustrates a side perspective view of one potential embodiment of a steam port device of the present invention in accordance with the disclosed architecture; and

FIG. 3 illustrates a perspective view of one potential embodiment of a steam port device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an improved steam port device. There also exists a long-felt need in the art for a steam port device that provides users with a commercial steam cleaning system capable of being used to clean and disinfect the interior of a vehicle. In addition, there exists a long-felt need in the art for a steam port device that allows users to use a steam wand and squeeze the handheld trigger to dispense the selected steam cleaning cycle into the vehicle for disinfecting and cleaning the interior. Further, there exists a long-felt need in the art for a steam port device that has multiple attachments in order to properly reach certain areas of the vehicle as well as properly disinfect different interior vehicle surfaces.

The present invention, in one exemplary embodiment, is comprised of a steam port device. The device is primarily comprised of a body, at least one motor fixed inside of the body, and at least one steam wand. In the preferred embodiment, the body is comprised of at least one digital touch screen, at least one locking mechanism, at least one button, at least one payment panel, at least one water inlet, and at least one power cable. In a differing embodiment, the body may be comprised of a plurality of wheels. In this manner, the device may be rolled around by the user and made easily transportable.

The digital touch screen may be any touch screen known in the art that allows the user to access the different steaming options. In this manner, the user can easily see and select the different steaming options available to them when deciding which option is best for disinfecting their vehicle. The locking mechanism may be any locking mechanism known in the art that allows the body panel to be locked. In this manner, the locking mechanism can keep thieves from being able to steal money from the device. The button is fixed to the front of the body and allows the user to start the device.

The water inlet may be any inlet known in the art that can properly connect a water supply to the device. In this manner, the user can connect a water supply to the device to provide the device with water to produce the steam used for disinfecting and cleaning the vehicle. The power cable may be any power cable known in the art that can properly supply the correct amount of energy needed for the device to maintain proper function.

The payment panel is comprised of at least one credit card reader, at least one bill receiver, and at least one coin slot. The credit card reader can be any credit card reader known in the art that allows the user to make a payment. The bill receiver and the coin slot are fixed to the front of the payment panel. In this manner, the user has the option of three separate forms of payment when using the device.

The motor may be any motor known in the art that can generate enough heat to produce steam from the water supply. The steam wand is comprised of at least one hose, at least one trigger, at least one wand grip, at least one hose connector, and at least one wand storage. The hose carries the steam to the steam wand. In the preferred embodiment, the hose may be a rubber material that is preferably heat-resistant to protect against the high temperatures of the steam or any hose of the like. The hose attaches to the body of the device through a hose connector. The hose connector secures the hose to the body of the device and may be any connector known in the art that is heat resistant.

The trigger may be any trigger known in the art that causes the release of pressure. In this manner, the user can pull the trigger to release the steam from the wand to clean their vehicle. The trigger is fixed to the wand grip. In the preferred embodiment, the wand grip may be made of a textured, rubber material that is heat-resistant or any grip of the like. In this manner, the steam wand can be safely and securely held so as to not cause injury by the release of steam. The wand storage is fixed to the side of the body of the device and allows a safe storage location for the steam wand when the device is not in use.

In a differing embodiment, the steam wand is comprised of at least one wand adapter. The wand adapter may be any adapter known in the art that provides alternative cleaning capabilities while using the steam wand. In this manner, differing wand adapters can be used to access hard-to-reach places inside the vehicle. The wand adapter is comprised of at least one opening that allows the wand adapter to fit onto the steam wand.

Accordingly, the steam port device of the present invention is particularly advantageous as it provides users with a commercial steam cleaning system capable of being used to clean and disinfect the interior of a vehicle that includes a steam wand and hose for improved accessibility so that the user does not need to strain themselves during the cleaning process. In this manner, the steam port device overcomes the limitations of existing steam cleaners known in the art.

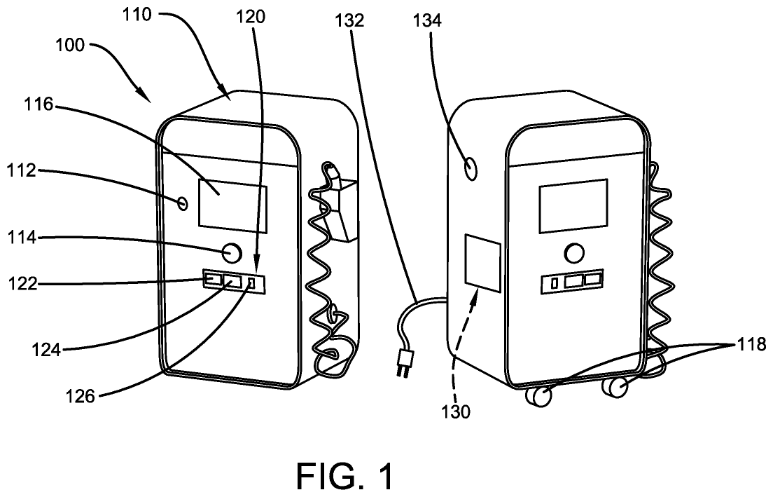

Referring initially to the drawings, FIG. 1 illustrates a front perspective view of one potential embodiment of a steam port device 100 of the present invention in accordance with the disclosed architecture. The device 100 is primarily comprised of a body 110, at least one motor 130 fixed inside of the device 100, and at least one steam wand 140. In the preferred embodiment, the body 110 may be manufactured from a metal material such as, but not limited to, sheet metal, stainless steel, galvanized steel, black steel, black iron, brass, nickel alloys, carbon steel, duplex steel, copper nickel, titanium, cast iron, aluminum, etc. In differing embodiments, the body 110 may be manufactured in a plurality of shapes such as, but not limited to, square, rectangle, triangle, circle, oval, etc., or any shape that allows the device 100 to house all of the internal components. In a differing embodiment, the body 110 may be comprised of at least one wheel 118 such that the body 110 can be easily moved.

In the preferred embodiment, the body 110 is comprised of at least one digital touch screen 116, at least one locking mechanism 112, at least one button 114, at least one payment panel 120, at least one water inlet 134, and at least one power cable 132. The digital touch screen 116 can be any digital touch screen known in the art such as, but not limited to, a 5-wire resistive touch screen, a surface capacitive touch screen, a projected capacitive touch screen, a surface acoustic wave touch screen, an infrared touch screen, etc. The digital touch screen 116 allows the user to select the proper steam cycle of the motor 130. Upon selection, the button 114 can be pressed to start the steam cycle, which can persist for a period of time. The button 114 can be any button known in the art.

In the preferred embodiment, the locking mechanism 112 may be any mechanism such as, but not limited to, a clamp, a keylock, a magnet, a combination lock, a padlock, etc. The locking mechanism 112 prevents the body 110 from being opened and therefore deterring theft.

In the preferred embodiment, the power cable 132 may be any type known in the art. However, the cable 132 is preferably a 15-watt amp cord. In a differing embodiment, the power cable 132 can be any power cable known in the art which can provide enough power to the motor 130. In the preferred embodiment, the water inlet 134 can be any inlet known in the art that provides a secure, water-tight connection to supply water into the body 110.

The payment panel 120 is comprised of at least one credit card reader 122, at least one bill receiver 124, and at least one coin slot 126. The credit card reader 122 can be any credit card reader known in the art such as, but not limited to, a magstripe reader, a chip reader, an EMV reader, a contactless card reader, etc. The bill receiver 124 is positioned between the credit card reader 122 and the coin slot 126 as an alternative method of payment. In the preferred embodiment, the coin slot 126 can be an opening in the payment panel 120 so that the user can place coins inside as a form of payment. Once payment is received by the panel 120, reader 122, receiver 124 and/or slot 126, the motor 130 is powered on.

The motor 130 can be any motor known in the art that produces steam such as, but not limited to, a steam motor, a steam turbine, an external combustion engine, etc. The motor 130 is located inside the body 110 of the device 100.

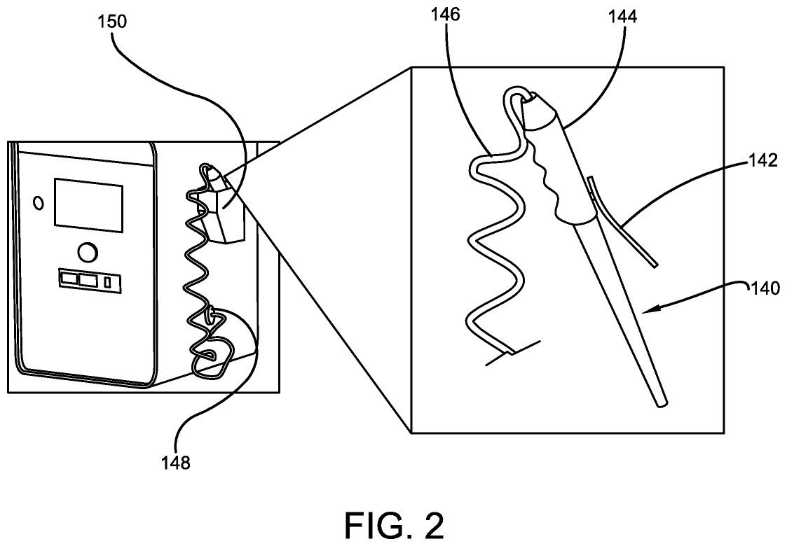

FIG. 2 illustrates a side perspective view of one potential embodiment of a steam port device 100 of the present invention in accordance with the disclosed architecture. The steam wand 140 is comprised of at least one hose 146, at least one trigger 142, at least one wand grip 144, at least one hose connector 148, and at least one wand storage 150.

In the preferred embodiment, one end of the hose 146 fixedly attaches to the steam wand 140 and carries steam to the steam wand 140 from the motor 130. The other end of the hose 146 fixedly attaches to the body 110 through the hose connector 148. In the preferred embodiment, the hose 146 may be a rubber material that is preferably heat-resistant to protect against the high temperatures of the steam or any hose of the like.

The hose connector 148 may be any connector known in the art that is heat resistant. In a differing embodiment, the hose connector may be manufactured from any heat-resistant, rigid plastic known in the art such as, but not limited to, acrylic, polycarbonate, polyethylene, thermoplastic, acrylonitrile butadiene styrene, low-density polyethylene, medium-density polyethylene, high-density polyethylene, polyethylene terephthalate, polyvinyl chloride, polystyrene, polylactic acid, acetal, nylon, fiberglass, recycled plastic, biodegradable plastic, etc.

At least one trigger 142 is fixedly attached to the wand grip 144. The trigger 142 may be any trigger known in the art that causes the release of pressure. In the preferred embodiment, the wand grip 144 may be made of a textured, heat-resistant material such as, but not limited to, a granulated grip, a rubber grip, a granulated/rubber combination grip, etc.

The wand storage area 150 is fixed to the side of the body 110 of the device 100 and allows a safe storage location for the steam wand 140 when the device 100 is not in use. In the preferred embodiment, the wand storage 150 may be manufactured from any heat-resistant, rigid plastic known in the art such as, but not limited to, acrylic, polycarbonate, polyethylene, thermoplastic, etc. In a differing embodiment, the wand storage 150 may be manufactured from a metal material such as, but not limited to, sheet metal, stainless steel, galvanized steel, black steel, black iron, brass, nickel alloys, carbon steel, duplex steel, copper nickel, titanium, cast iron, aluminum, etc.

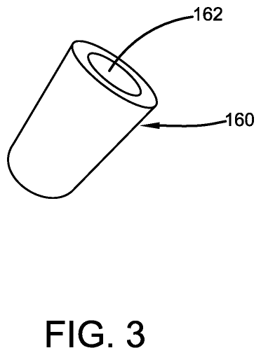

FIG. 3 illustrates a front perspective view of one potential embodiment of a steam port device 100 of the present invention in accordance with the disclosed architecture. In a differing embodiment, the steam wand 140 is comprised of at least one wand adapter 160. The wand adapter 160 may be any shape, size, and structure that provides alternative cleaning capabilities while using the steam wand 140. The wand adapter 160 may be manufactured from a metal material that can withstand the high temperatures from the steam. The wand adapter 160 is comprised of at least one opening 162 that allows the wand adapter 160 to fit onto the steam wand 140. In differing embodiments, the opening 162 may be of any diameter to allow the opening 162 to fit over the steam wand 140 of any size.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “steam port device” and “device” are interchangeable and refer to the steam port device 100 of the present invention.

Notwithstanding the foregoing, the steam port device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the steam port device 100 as shown in the FIGS. are for illustrative purposes only, and that many other sizes and shapes of the steam port device 100 are well within the scope of the present disclosure. Although the dimensions of the steam port device 100 are important design parameters for user convenience, the steam port device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A steam port device comprising:
a body;
a motor;
a steam wand comprised of a trigger;
a screen;
a credit card reader;
a bill receiver; and
a coin slot.

2. The steam port device of claim 1, wherein the motor produces steam.

3. The steam port device of claim 2, wherein the steam wand is in fluid communication with the motor via a hose.

4. The steam port device of claim 1 further comprised of a wheel.

5. A steam port device comprising:
a body;
a motor;
a steam wand comprised of a trigger;
a touch screen;
a power cord;
a credit card reader,
a bill receiver; and
a coin slot.

6. The steam port device of claim 5, wherein the motor produces a steam for a period of time.

7. The steam port device of claim 6, wherein the period of time can be selected by the touch screen.

8. The steam port device of claim 5, wherein the power cord is comprised of a 15-watt power cord.

9. The steam port device of claim 5, wherein the steam wand is in fluid communication with the motor via a hose.

10. The steam port device of claim 5, wherein the credit card reader is comprised of a EMV reader.

11. A steam port device comprising:
a body;
a wheel;
a hose;
a locking mechanism;
a motor;
a steam wand comprised of a trigger;
a touch screen;
a power cord;
a credit card reader;
a bill receiver; and
a coin slot.

12. The steam port device of claim 11 further comprised of a water inlet.

13. The steam port device of claim 11, wherein the motor is comprised of a steam motor.

14. The steam port device of claim 11, wherein the motor is comprised of a steam turbine.

15. The steam port device of claim 11, wherein the steam wand is in fluid communication with the motor via the hose.

16. The steam port device of claim 15, wherein the hose attaches to the body via a hose connector.

17. The steam port device of claim 16, wherein the hose connector is comprised of a heat-resistant material.

18. The steam port device of claim 11, wherein the wand is comprised of a grip.

19. The steam port device of claim 11 further comprised of a wand storage area positioned on the body.

20. The steam port device of claim 11, wherein the credit card reader is comprised of a chip reader.
